(19) United States
(12) Patent Application Publication
(10) Pub. No.: US 2015/0346182 A1
(43) Pub. Date: Dec. 3, 2015

(54) Portable System and Method for Quality Assurance Testing of Asphalt Binders

(71) Applicant: The Board of Trustees of the University of Illinois, Urbana, IL (US)

(72) Inventors: Jagannath Mallela, Champaign, IL (US); Samuel H. Carpenter, Champaign, IL (US)

(21) Appl. No.: 14/294,819

(22) Filed: Jun. 3, 2014

(51) Int. Cl. G01N 33/38; G01L 1/22

(52) U.S. Cl. CPC G01N 33/383; G01L 1/22

(57) ABSTRACT

A system includes a voltage supply configured to supply a drive voltage. A flexible device connects with the voltage supply. The flexible device is configured to be embedded into a material, e.g., a viscoelastic material. A strain gage connects with the flexible device, and the strain gage is configured to measure a flexing of the flexible device. A processor connects with the strain gage. The processor is configured to determine a measured strain and a phase shift between the drive voltage and the strain when voltage is supplied to the flexible device to produce a fingerprint of the material. The processor can be configured to determine G* and δ to produce a fingerprint of the material.

### BACKGROUND

The growth of the automobile created a need for more paved surfaces. A need was also created for roads that had tougher and more resistant surfaces than their macadamized counterparts. The asphalt supply from the natural sources could not meet these demands. Fortunately, concurrent to the demand for better road wearing surfaces was the demand for increased gasoline production for the automobiles. This latter demand led to the development of modern day paving asphalt, which is a by-product petroleum crude distillation process through which gasoline is manufactured. This form of asphalt did not change greatly in composition until the 1980s when additives such as tire rubber, polymers, and other chemicals began to be added in order to improve the fatigue and low-temperature properties. What has changed in asphalt production over the years is the refining process, material handling and distribution and asphalt grade specification.

### BRIEF DESCRIPTION OF THE DRAWINGS

In association with the following detailed description, reference is made to the accompanying drawings, where like numerals in different figures can refer to the same element.

FIG. 1 is a top view of an exemplary asymmetrically poled duomorph device showing power and ground leads, a grounding tab and strain gage affixed to its geometric center.

FIG. 2 is a section view of an exemplary device for testing asphalt binders showing the asymmetric poling axes of the piezoceramic sheets.

FIG. 3 is an exemplary side view of the device in operation.

FIG. 4 is an exemplary schematic of the device in operation, illustrating determination of signal shift and gage strain.

FIG. 5 is an exemplary flow diagram of potential sampling locations for quality assurance testing of asphalt binders by the device.

FIG. 6 is a block diagram of a schematic of an exemplary system for operating the device.

FIG. 7 is a chart of exemplary peak piezo actuator current requirement for various devices.

FIG. 8 is a front view of an exemplary guide sleeve for positioning the device in asphalt binder.

FIG. 9 is a chart showing an exemplary non-dimensional voltage output of the device in an unaged AC-20 asphalt binder.

FIG. 10 is a chart showing an exemplary phase shift between the strain gage and drive signals of a device in air and in an unaged AC-20 binder.

FIG. 11 is a chart showing an exemplary strain ratio signatures produced by the device in various unaged and aged asphalt binders when operated at a frequency of 1.59 Hz.

FIG. 12 is a chart showing an exemplary strain ratio versus non-dimensional stiffness term, M′, for various devices when embedded in the AC-20 binder.

FIG. 13 is a chart showing exemplary dimensions and flexural rigidities of different devices.

FIG. 14 is a chart showing an exemplary strain ratio signatures produced by the device in various unaged and aged asphalt binders when operated at a frequency of 1.59 Hz.

FIG. 15 is an exemplary graph showing a correlation of a device measured strain ratio with the strain ratio estimated by a finite element model mimicking the device operation.

FIG. 16 is an exemplary graph showing a correlation of a device measured phase shift with the phase shift estimated by a finite element model mimicking the device operation.

FIG. 17 is a chart of an exemplary comparison of strain ratios from the finite element model runs and the regression equations that fit the data from the finite element model runs.

FIG. 18 is a chart of an exemplary comparison of phase shifts from the finite element model runs and the regression equations that fit the data from the finite element model runs.

FIG. 19 is a chart of an exemplary nomograph for one of the devices showing the results of data reduction to convert measured strain ratios and phase shifts into complex shear modulus (G*) and phase angle (δ).

FIG. 20 is a chart of an exemplary comparison of DSR measured and the device estimated shear modulus (G*) at 1.59 Hz.

FIG. 21 is a chart of an exemplary comparison of DSR measured and the device estimated phase angle (δ) at 1.59 Hz.

### DETAILED DESCRIPTION

A system, method and device are disclosed to rapidly and cost-effectively test polymers, e.g., virgin and modified asphalt binders, for specification compliance at the refinery, the blending terminal, the asphalt plant, and at the job site. The device can be used to replace or supplement the Dynamic Shear Rheometer (DSR) testing and can be used to directly estimate the AASHTO M320 Table 1 specification parameters, e.g., the complex shear modulus (G*) and phase angle (δ) over a portion of the pavement in-service temperature range the binder is expected to perform under. The raw device responses—strain ratio and phase and strain ratio—are unique to each asphalt binder grade tested which can be used to “fingerprint” the asphalt binders and function as an effective quality assurance (QA) tool. These responses can be used in conjunction with a finite element (FE) based nomographical solution scheme to determine G* and δ of the asphalt binder thus providing another level of fingerprinting. Since asphalt binders manufactured from various sources vary in chemical and physical properties, the device can thus be used for rapid quality control and verification testing of asphalt binders along the asphalt binder's journey from a supplier's tank where it is certified to the job-site in a cost-effective and rapid manner. The systems, methods and devices can be used to standardize gages to test asphalt binders over the range of asphalt stiffness where it is expected to produce high quality outputs. The device can be used to provide good outputs over a reasonable range of asphalt binder stiffness. A housing assembly for the gages and the various lead wires can allow it to operate efficiently over an extended period of time without breaking down.

FIG. 1 is a top view of an exemplary asymmetrically poled duomorph device showing power and ground leads, a grounding tab and strain gage affixed to its geometric center. The device can include a metallic grounding tab to gain access to the central metallic shim which is grounded. The tab can be triangular shaped and sized to have enough space to help make an electrical ground connection. Other shapes can be used. The piezoelectric material on either sides of the tab are filed until the central metallic shim is exposed. A ground lead is connected, e.g., soldered to the tab and used to provide an access point for the electrical ground lead. An advantage of grounding the device with the tab is to allow both sides of the device to be driven simultaneously to produce higher deflections for a given input voltage when compared to a device whose piezoelectric faces are driven with respect to each other. For example, asymmetrically poled devices with both PZT layers driven with respect to the grounded center metallic shim can increase, e.g., about double, the output from the same applied input.

Although the tab can introduce some asymmetry in the device geometry, the tab dimensions are small enough to prevent it from being considered as an axisymmetric solid of revolution. Power leads connect to a strain gage located on the device. In one example, the ground lead and the power leads are 16 gauge and connected to the device by solder. The strain gage can include bonded resistance foil strain gages, bonded semiconductor strain gages, etc. Use of the strain gage in lieu of piezosensing are intended to reduce the effects of signal drifts due to variations in testing temperature or due to static preload when the device is embedded in asphalt at lower temperatures. In other implementations, the piezoelectric can be used as both an actuator and a sensor. Strain gages from Micro Measurements, Inc., of the type EA-06-125AC-350 with a gage length of 0.122 in. can be used. Further modifications to the device can include arranging the piezoelectric layers with their poling axes asymmetrically placed about the neutral axis. This arrangement can allow both the faces of the device to be driven simultaneously with a voltage signal of like polarity effectively doubling the strain output. Alternatively, one face of the device can be driven with respect to the other face which is grounded resulting in a symmetric device where one-half of the net piezoelectric potential is utilized. The device with the highest flexural rigidity, has the lowest voltage or moment ratio, and vice versa.

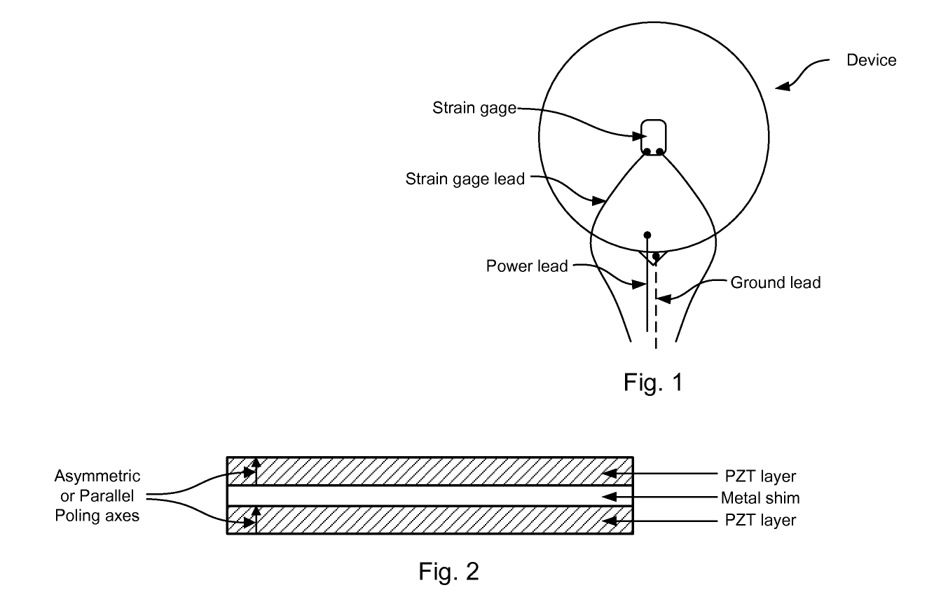

FIG. 2 a section view of an exemplary device for testing materials, e.g., for testing polymers present in asphalt binders showing the asymmetric poling axes of the piezoceramic sheets. The device can include a sandwich assembly of a circular steel disc or shim placed concentrically between two piezoelectric circular discs. Other materials for the shim can be used, e.g., silver, brass, etc. In other implementations, the device can be known as a duomorph, duomorph gage or sensor, bimorph or a piezoelectric bender. The operational characteristics of the device are influenced by the geometric properties of the individual components of the device as well as their materials properties, e.g., the piezoelectric discs.

The piezoelectric element of the device can be given a preferential mode of operation, e.g., elongation, flexure, or shear depending upon the applied electrical field/charge or mechanical strain/stress, the orientation of the polarization axes, stacking of the individual piezoelectric layers, etc. The piezoelectric material can be manufactured from ceramics (e.g., PZT-4, PZT-5A, PZT-5H, etc.), plastics, quartz, Rochelle salt, tourmailine, etc. In one implementation, the PZT-5A is a piezoceramic material bearing the trade name PSI-5A-S2 from Piezo Systems, Inc., Cambridge, Mass. is used. The material can be brushed nickel electroded on either side. The methods, systems and devices described herein utilize the flexurally response of the piezoelectric elements. Other piezoelectric elements that qualify as flexure mode transducers or gages can be used, including unimorhps, duomorphs, multimorphs and other multi-electroded, single-poled, specially treated single-plate elements. In one implementation, the overall thickness of the device is about half a millimeter. The different layers in the device can be held together by a layer of a special bonding agent.

The exposed surfaces of the piezoelectric layers can be coated finely with electrodes (silver, nickel, etc.) to enable electrical charge. Electrical leads are soldered to the surface of these electroded faces to facilitate electrical excitation or measurement. The electrical leads can be connected with the surfaces in other ways. The piezoelectric sheets can be thickness poled and arranged either in series, e.g., both poling axes point in the opposite directions, or parallel, e.g., both poling axes point in the same direction, about the neutral axis of the device assembly. The choice between series and parallel type can depend on individual factors discussed more below. The device as shown is a circular disc shaped bending element, but other shapes, e.g., oval, square, rectangular, etc., may be used. If this is done, a new nomographical solution can be developed to reduce the raw device outputs, e.g., strain ratio and phase shift, to G* and δ.

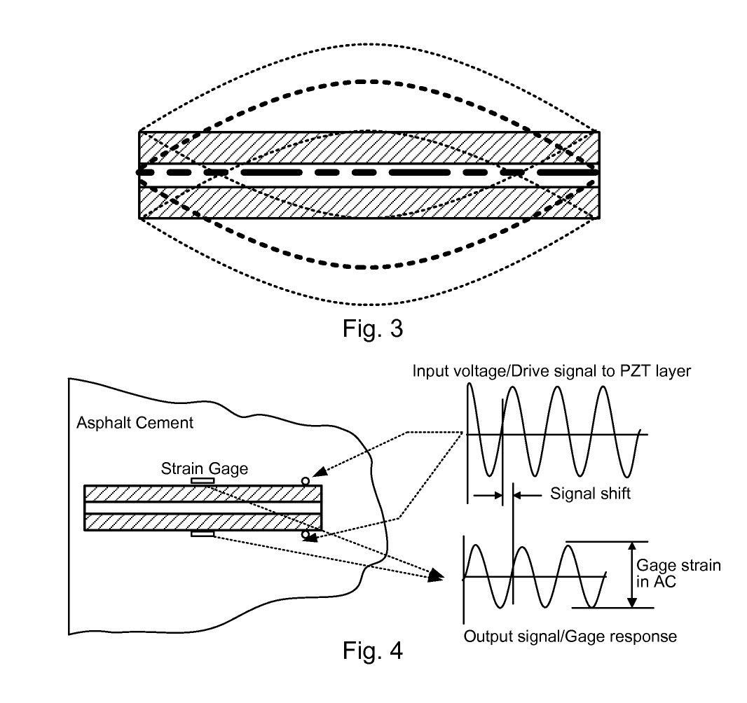

FIG. 3 is an exemplary side view of the device in operation. The piezoelectric layers function as electromechanical transducers capable of producing a mechanical deformation when an electrical voltage is applied to them, and vice versa. When a voltage is applied to a PZT crystal it deforms, e.g., expands or contracts within the embedded material, e.g., asphalt binder. In an electrically asymmetric device, the poling axes of the two piezoelectric layers are oriented such that, when a voltage is applied across their electroded faces, one of the layers expands while the other contracts. In such parallel type devices the electroded piezoelectric faces are driven simultaneously with a voltage of like polarity with respect to an electrical ground contact established with the metal vane. This produces a bending action as indicated in the section view of the device as shown. The maximum bending strain occurs at the center of the device. Therefore, this is a location for strain gaging and measuring a device response within the embedded material. Other types of measurement can be made, e.g., direct measurement of the piezoelectric electrical response. Also, an electrically symmetric or series type devices can be used instead of the asymmetric device. The term symmetric can include that the poling axes of the PZT layers in the device point in opposite directions about the neutral axis, e.g., they point away from each other or toward each other. Asymmetric devices are those where the poling axes of the PZT layers point in the same direction.

The magnitude of the bending strain at the device center is directly proportional to the driving voltage. The amount of bending can increase linearly with the applied voltage. Higher driving voltages can be required to obtain cleaner signals, e.g., lower signal-to-noise ratio, particularly when the device is embedded in a viscoelastic medium, e.g., asphalt. The driving voltage does not cross the depolarization limit so that the piezoelectric properties are not lost.

If the applied voltage signal is sinusoidal, the device vibrates sinusoidally at the same frequency as that of the input signal. When the device is operated in air, the strain signal can follow the trace of the driving voltage, e.g., the time lag or signal shift is zero. This provides a calibration point for analysis when the device is embedded in the material, e.g., the asphalt binder. When the device is embedded in a viscoelastic medium and vibrated, two changes occur to the signal. There can be a time lag induced in the response of the device with respect to the applied driving voltage signal, and the peak strain can be reduced due to the confining effect of the stiffness of the surrounding medium. The signal phase shift along with the ratio of peak device strains in air and in the medium (strain ratio) provides a way to compute the properties of interest of the surrounding medium. For the case of asphalt materials properties of interest include the G* and δ of the viscoelastic medium.

FIG. 4 is an exemplary schematic of the device in operation, illustrating determination of signal shift and gage strain. The shift in the bending strain response is not the same as the phase angle, δ, of the asphalt binder measured using the DSR. Instead, phase angle is one of the parameters on which the signal shift is dependent upon. Other factors that influence this parameter include the stiffness of the surrounding medium and the geometric and material properties of the materials of the device. Factors include PZT layer thickness, types of backing materials, e.g., steel, brass, and no shim, thickness of the backing material, e.g., 0.004, 0.005 and 0.008-in, thickness of the PZT material, e.g., 0.005 and 0.0075-in, a diameter of the device, e.g., 0.5 in, 0.75 in, 1 in, and 2 in., and a poling axis orientation of PZT layers with respect to neutral axis, e.g., symmetric or series poled and asymmetric or parallel poled. Fexural rigidity of the device can be determined as:

$$D = \frac{{E}_{z} {h}^{3}}{12 ( 1 - {v}^{2} )} [ 1 + ( \frac{{E}_{m}}{{E}_{z}} - 1 ) {( \frac{{h}_{m}}{h} )}^{3} ] \qquad ( 1 )$$

where D is a gage flexural rigidity, E_{z} is the modulus of the PZT disk, Eₘ is the modulus of the metal disk, hₘ is the thickness of metal disk, and h is the overall thickness of the device. A more sensitive device, e.g., one which would produce more deformation per volt, is used for the elevated testing temperatures when the device is embedded in asphalt. The sensitive shim-based devices can be made with a thin backing material, e.g., made out of brass or stainless steel. A thin shim made with a lower modulus metal can increase the device sensitivity by decreasing the lower overall flexural rigidity of the device. In addition, a shimless device, e.g., a device with no metallic shim in the center, can also be used. Furthermore, piezoplastics can be used in lieu of piezoceramics as the choice piezoelectric materials.

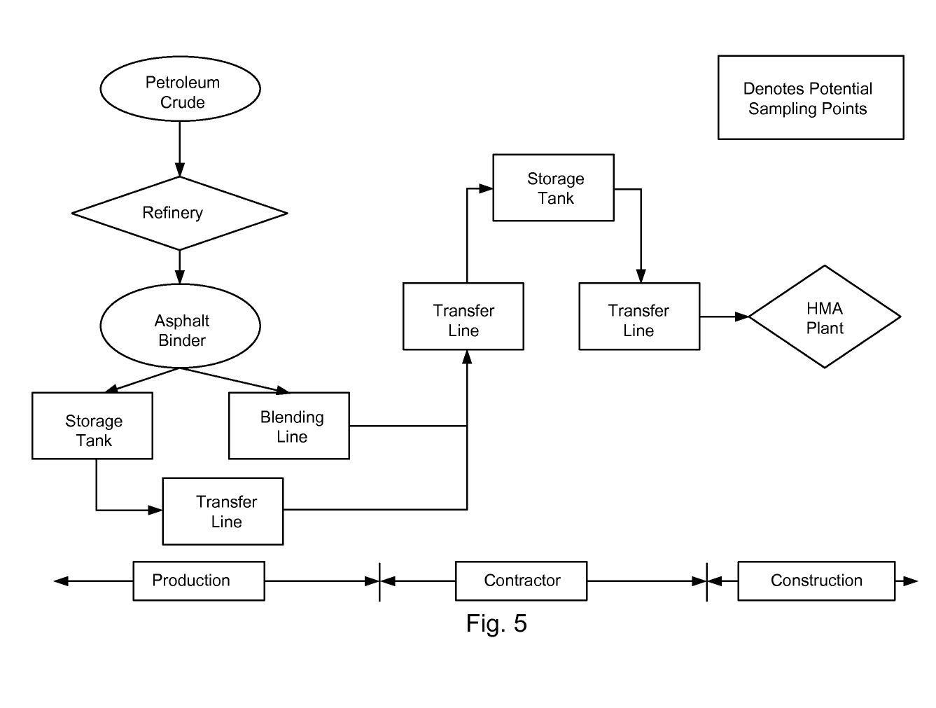

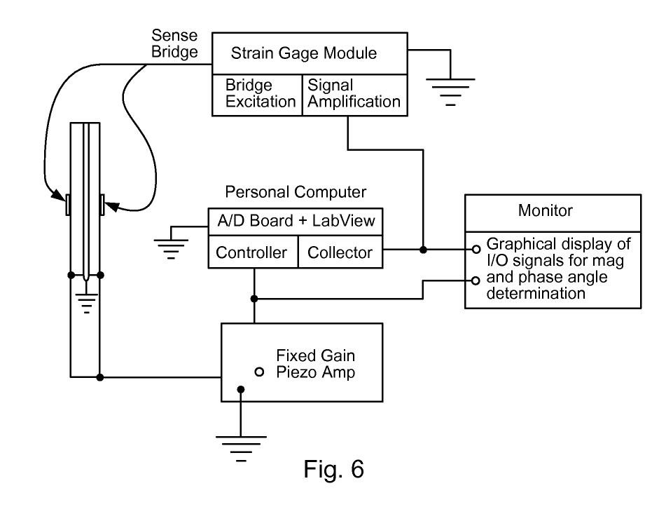

FIG. 5 is an exemplary flow diagram of potential sampling locations for quality assurance testing of asphalt binders by the device. The device can test asphalt binders at any of the production stage, contractor stage and construction stage. For example, at the production stage, the petroleum crude is sent to the refinery to become an asphalt binder. The asphalt binder can be tested at any of storage tanks, blending lines, and transfer lines. At the contractor stage, the device can be used to test the asphalt binder at the transfer lines and storage tanks. The device can also be used to test the asphalt binders at construction site plants. Factors that can affect the asphalt binder consistency at these different stages include storage temperature, e.g., overheating, blending, changing crude sources, the refinery process, e.g., temperature and pressure, contamination in tanks, storage time, separation, dilution, the presence of modifiers, etc.

The portability and ease of use of the device can accommodate testing the asphalt binders at these different locations. This is in contrast to other ways of testing, including the rolling thin film oven (RTFO) test, the pressure aging vessel (PAV) test, the dynamic shear rheometer (DSR) test, the bending beam rheometer (BBR) test, and the direct tension (DT) device. Unlike these tests, the device does not require a controlled laboratory environment, the device can be handheld and device readings can be performed in the field. The device does not require a significantly skilled operator, the cost of the device equipment can be lower than the other devices and does not require maintenance by qualified staff, the device can be used with crumb rubber modified binders that exceed the gap setting of the device's parallel plates.

Error! Reference source not found. is a block diagram of a schematic of an exemplary system for operating the device. The electronics include a personal computer equipped with a 16 channel, National Instruments AT-MIO-16E-2 analog-to-digital converter (ADC) board. The functions of the computer and board can include generating the drive signal, e.g., drive voltage, which produces deformations in the device, conditioning the strain gage signals from the device as it deflects into a form that can be converted to digital values, and converting the conditioned sensor signals to digital values. A precision external piezo linear amplifier can amplify the voltage signal from the ADC board for driving the device. The peak current requirements of piezo actuator can be determined as Iₚ=2*π*f*C*Vₚ, where Iₚ=Peak current, Amperes, f is the maximum operating frequency, Hz, C is the capacitance of the piezo device, Farads, and Vₚ is maximum peak voltage required by the piezo actuator, Volts.

The device assembly can include a signal conditioning and amplification module. At the lower temperatures, the strain output from the device is very small and can require amplification of up to ×2000 for the given driving voltages and sensitivities of the strain gages used. A Measurements Group® Vishay 2000 unit can provide the signal amplification. The Vishay unit has two internal 350-ohm resistance gages and completes the Wheatstone bridge circuit along with the two strain gages affixed to the device. Both quarter- and half-bridge circuits can be used. The bridge excitation can be varied from 10 to 15 VDC using a voltage supply, to determine the optimal value for the various asphalt stiffnesses considered.

Virtual instruments or VIs, e.g., developed using the National Instruments LabVIEW software, can drive the device and store the resulting input and output waveforms, e.g., in a spreadsheet format. The VIs developed include test actuation VI to simulate the sunisoidal waveform, its frequency, and amplitude to actuate the load pulse and data acquisition VIs to acquire temperature and strain outputs data from sensor attached to the device. The VI can filter the signal, analyze the signals, e.g., to determine amplitude and store the signal data. Peak strains and lag between the input and output signals, e.g., the desired inputs to the data reduction scheme discussed later, e.g., can be derived from the data stored and further processed to obtain the test parameters of interest.

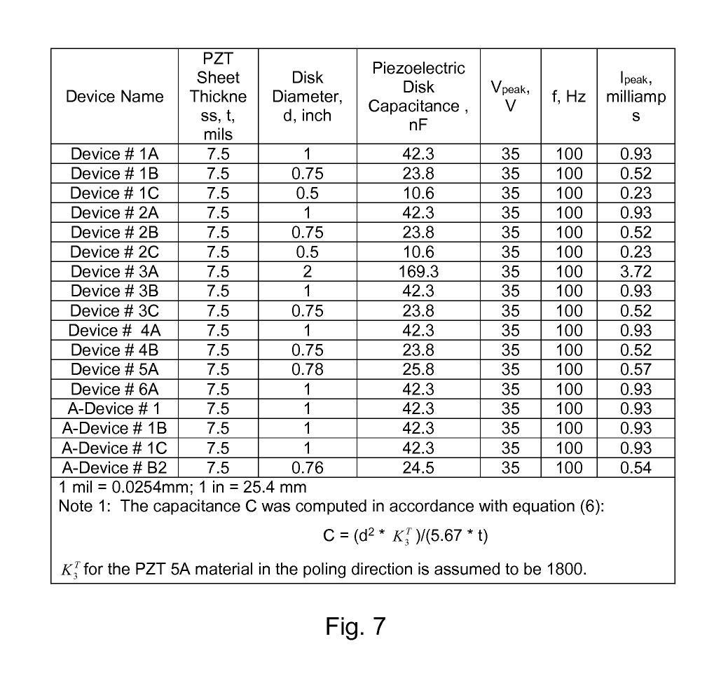

FIG. 7 is a chart of exemplary peak piezo actuator current requirements for various devices. The current requirements to drive the actuator are under 1 milliamp for a majority of the configured devices and less than 4 milliamps for the largest diameter device assembled. The amplifier can supply the peak voltage and current over the desired actuation frequency range. The strain gage can pick up signals from the device which can be amplified and sent to a monitor for graphical display of the input/output (I/O) signals for magnitude and phase angle determination. The output signals can also be collected by the A/D board to be processed and used to control the fixed gain piezo amplifier connected with the load lead inputs of the device. A combination of the test control and data acquisition hardware help produce a digitally signal with precise control. The device labelled A-Device #1C is the device implemented for the discussion herein.

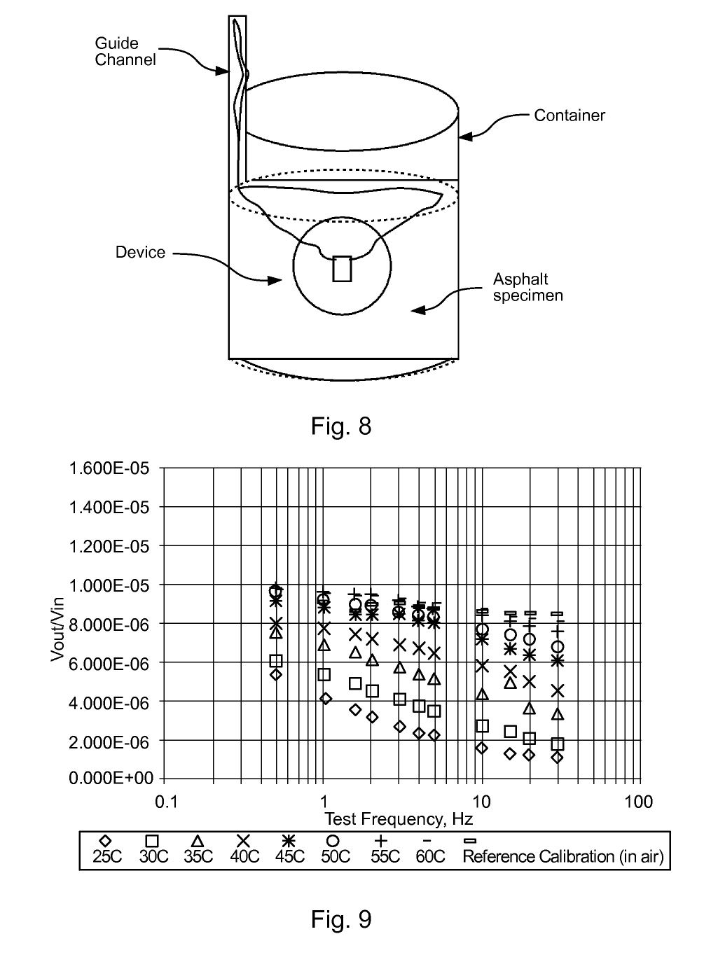

FIG. 8 is a front view of an exemplary guide sleeve for positioning the device in the material, e.g., asphalt binder, for fingerprinting. The guide sleeve may be manufactured from metal or other sturdy material. The guide sleeve includes a guide channel to house the leads of the device. A bottom of guide sleeve can rest on a bottom of the container holding the asphalt specimen and position the device in the center of the asphalt specimen. An exemplary container for holding the asphalt binder to be tested is four inches tall by four inches in diameter. In addition to facilitating device placement, the guide sleeve can also help haul the device out of the asphalt specimen without pulling directly on the wire leads attached to the device. The flexing of the device is pushed against by the material in which the device is embedded to test the rheology or deformation and flow of material, e.g., the non-Newtonian flow of liquids and the plastic flow of solids, for fingerprinting the material.

Asphalt binder temperatures can range, for example, from approximately −10° C. (14° F.) to approximately 60° C. (140° F.). Non-dimensional voltage can be determined as:

$$\frac{{V}_{out}}{{V}_{im}} = ( \frac{{V}_{out}^{AC}}{{A}_{AC} * {V}_{in}^{AC}} ) ( \frac{{V}_{out}^{air}}{{A}_{air} * {V}_{in}^{air}} ) \qquad ( 2 )$$

where,

$$\frac{{V}_{out}}{{V}_{im}}$$

is the non-dimensional output voltage, Vₒᵤₜ^{AC} is the output of strain gage affixed to the device embedded in asphalt, V, A_{AC} is the applied gain to a strain gage affixed to the device embedded in asphalt, Vᵢₙ^{AC} is the drive voltage applied to the device embedded in asphalt, V, Vₒᵤₜ^{air} is the output of strain gage affixed to the device operated in air, V, Aₐᵢᵣ is the applied gain to a strain gage affixed to the device operated in air, and Vᵢₙ^{air} is the drive voltage applied to the device operated in air, V.

FIG. 9 is a chart showing an exemplary non-dimensional voltage output of the device in an unaged AC-20 asphalt binder. An output of the device is sensitive to the asphalt binder stiffness with the stiffer binders causing a lower non-dimensional voltage output (and hence strain) and vice versa at any given frequency. The output of the device decreases as the test frequency increases. As the test temperature goes from low to high, the gage output increases approaching an unconstrained value established by testing in air. For example, as binder temperature approaches 60° C. (140° F.), the strain gage behaves as if it is in air thus, e.g., loses sensitivity to the surrounding asphalt binder. This can establish the limiting lower binder stiffness over which the strain gage is useful. A stiffness of the device can be matched to the stiffness of the test medium to produce results across the stiffness ranges of interest to binder testing.

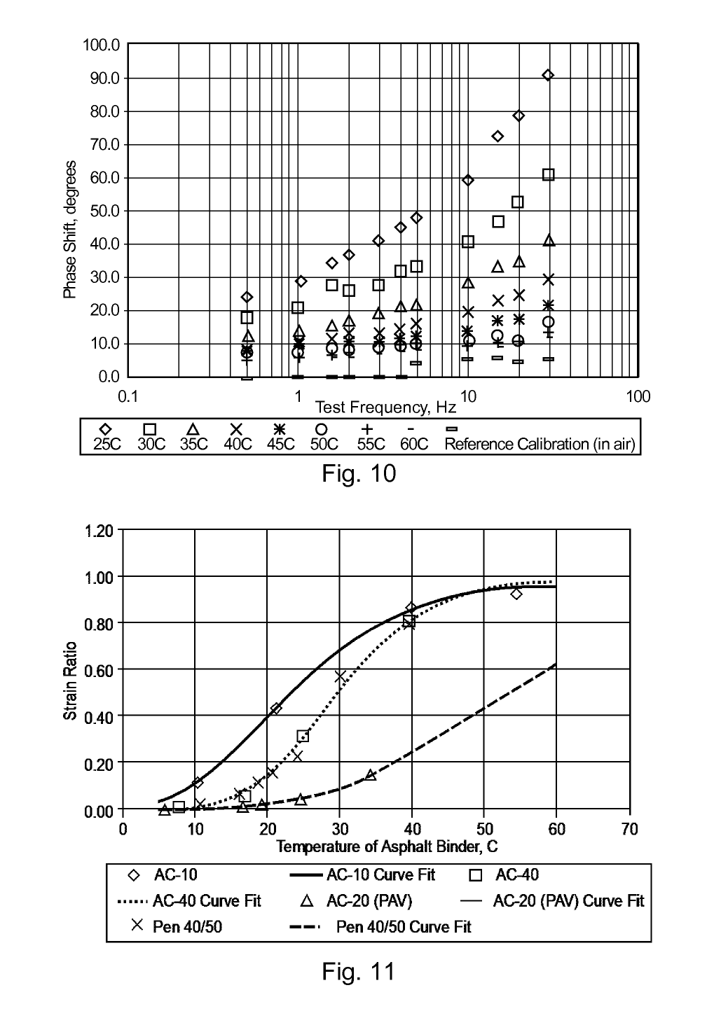

FIG. 10 is a chart showing an exemplary phase shift between the strain gage and drive signals of a device in air and in an unaged AC-20 binder. The phase shift decreases as the asphalt binder temperature (and hence stiffness) decreases at any given test frequency. As the test frequency increases, the phase shift increases. As with the device's output voltage, the phase shift, when tested in asphalt can approach the corresponding test values in air as the test temperature approaches 60° C. (140° F.). FIGS. 9 and 10 establish the usefulness of the device to be used to fingerprint an asphalt binder over a range of temperatures and frequencies.

For low temperature testing, to increase the device output, e.g., more gage deflection and hence strain per unit of drive voltage, both sides of the device can be driven simultaneously. When both sides are driven, the power requirements can increase due to the 4:1 reduction in the electrical impedance, but the device draws lower current even at the upper end of frequencies of interest to asphalt binder testing. To drive both faces of the device, an asymmetric or parallel poled PZT material can be used, e.g., A-Device#1C. Devices with higher non-dimensional voltage ratios can be desirable because they produce a better quality signal and are able to exercise the asphalt specimen into which they are embedded more strongly to facilitate more precise data acquisition and ensure that the device can be used over a wide range of asphalt stiffnesses. The non-dimensional voltage ratios are generally inversely proportional to their flexural rigidities. The higher the flexural rigidity of the device, the lower the device's 100 output and vice versa.

FIG. 11 is a chart showing an exemplary strain ratio signature produced by the device when various unaged and aged asphalt binders are tested with it at a frequency of 1.59 Hz. The strain ratios were plotted as a function of a non-dimensional stiffness parameter, M′. This quantity represents the ratio of the asphalt binder and device stiffness

$${M}^{′} = \frac{{E}^{′} {a}^{3}}{D} \qquad ( 3 )$$

where, M′ is the asphalt binder-to-device non-dimensional stiffness, a is the radius of the device inpsi, E′ is the real part of the complex modulus of asphalt in psi, and D is the flexural rigidity of the device in lbf-in. The curves shown are for different asphalt binders. The softer asphalt produces a plot with a higher slope than the harder asphalts. The strain ratio approaches a value of unity much quicker for the softer binders when compared to stiffer asphalts. Such plots carried out over a range of frequencies can be used in field verification of an expected fingerprint of the asphalt binder material pre-established at the supplier location.

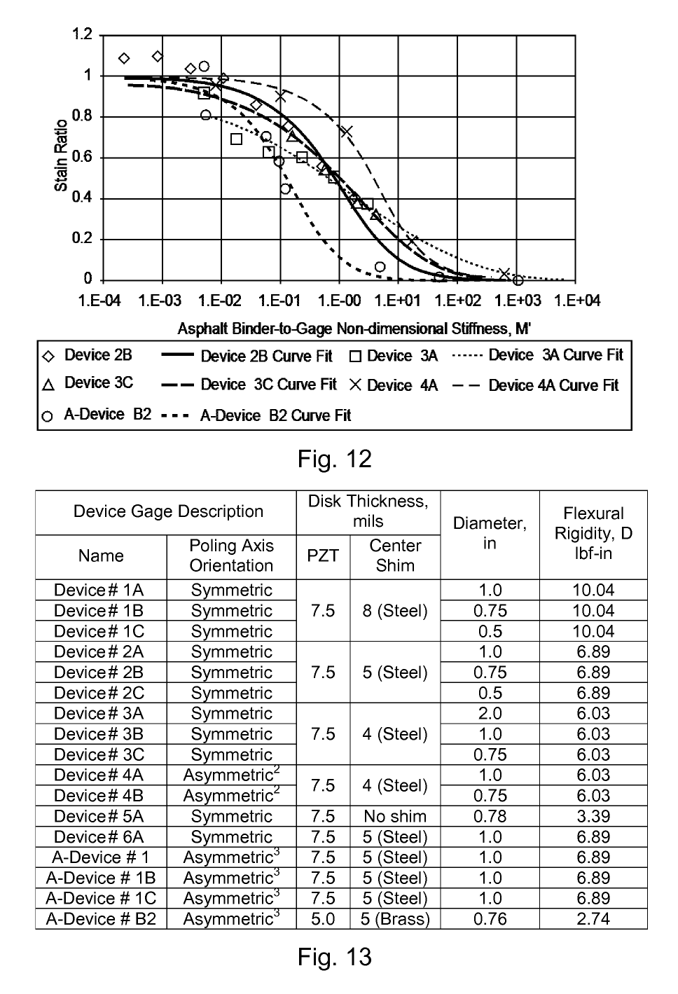

FIG. 12 is a chart showing an exemplary strain ratio versus non-dimensional stiffness term, M′, for various devices when embedded in the AC-20 binder and FIG. 13 is a chart showing exemplary dimensions and flexural rigidities of different devices. The FIG. 12 plot can be used to determine the allowable non-dimensional stiffness ratios to obtain desirable strain ratios, e.g., strain ranges that can be collected effectively and efficiently with the different devices. The device radius and rigidity can be varied to obtain a valid device for a given asphalt binder stiffness range of interest.

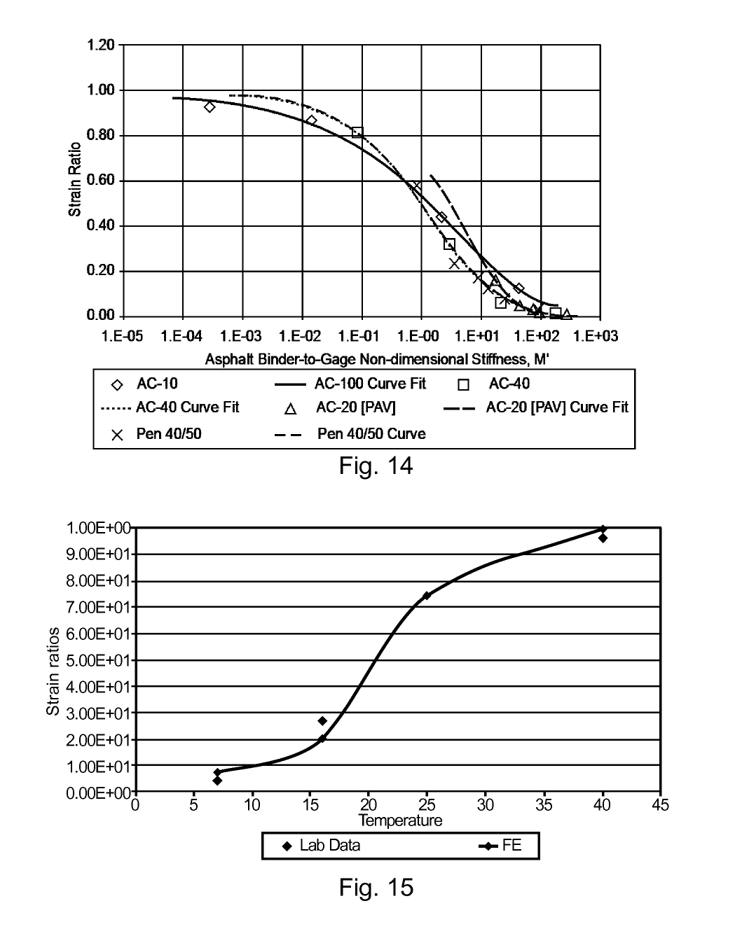

For a given device, as the asphalt binder gets softer, e.g., either due to elevated test temperatures or testing at lower frequencies, M′ decreases and vice versa. In general, matching the stiffnesses of the device and the surrounding asphalt binder to produce a gradual slope of the strain ratio-M′ curve results in a discriminating and accurate measurement environment. Avoiding strain ratios close to 1.0 and below 0.01 would avoid extreme M′ values and associated data gathering errors. Devices can lose sensitivity to the asphalt binder when the strain ratios approach values of 0.9. On the lower end, strain ratios below 0.01 can result in small strain readings of the device when embedded in asphalt (less than 1 micro strain) increasing the potential for measurement error. If these values of strain ratios are considered to provide the upper and lower bounds of the acceptable range of desirable outputs from the devices, then the corresponding M′ parameters for effective measurement can be determined for the various devices from FIG. 12 and FIG. 14. FIG. 14 is a chart showing an exemplary strain ratio signatures produced by the device in various unaged and aged asphalt binders when operated at a frequency of 1.59 Hz.

In one example, the device is a one inch (25.4 mm) diameter asymmetric device having a PZT thickness of 7.5 mils (0.19 mm) and a stainless steel center shim of 5 mils (0.13 mm), and a flexural rigidity, D, of 6.89 lbf-in, PZT layer elastic modulus of 8.27×10⁶ psi (57 GPa), PZT layer Poisson's ratio of 0.3, stainless steel elastic modulus of 30×10⁶ psi (207 GPa), and stainless steel Poisson's ratio of 0.3 (A-DEVICE#1C of FIG. 13). To establish ranges of asphalt binder stiffness over which the device can be expected to perform well, the real part of binder's complex shear modulus, G′, can be expressed approximately as:

$${G}^{′} = \frac{{E}^{′}}{2 ( 1 + v )} = \frac{{DM}^{′}}{{a}^{3}} \qquad ( 4 )$$

where, E′ is the real part of the complex modulus of asphalt, psi, ν is Poisson's ratio of asphalt binder taken to be 0.5, M′ is asphalt binder-to-device non-dimensional stiffness, a is the radius of the device, psi, and D is the flexural rigidity of the device, lbf-in. Using low and high values of M′ (0.004 and 231, respectively) with D and a of the A-DEVICE #1C, the range of G′ over which the gage can be effective when the binder stiffness is estimated to be 0.1 psi (0.69 kPa) to 4250 psi (29 MPa) and a phase angle between 30° to 75°, which can capture a majority of neat, modified, aged and unaged binders.

The following testing sequence can be followed when testing in an asphalt binder to establish a fingerprint for the asphalt binder. Heat the asphalt specimen to about 140° C. (284° F.) for about 5 minutes to allow the device to be inserted. Insert the device with the help of the guide sleeve into the asphalt specimen so that the gage is close to the center of the specimen. Place the specimen with the device into a temperature control chamber. Bring the specimen to the desired test temperature and take observations for each frequency of interest. Repeat for all test temperatures of interest. The test temperatures can include 5° C. (41° F.), 10° C. (50° F.), 20° C. (68° F.), 40° C. (104° F.), and 55° C. (131° F.). At each temperature, the asphalt specimen can be tested at 0.1, 1, 1.59, 10, 30, and 100 Hz. This testing can characterize the variability of the device output during a single sitting of frequency and temperature sweep analysis of an asphalt binder. An exemplary mean coefficient of variation (COV) of the device measurements in asphalt averaged over the entire range of test temperatures is 2.3 percent and the mean standard deviation is 0.072 V. An exemplary mean COV of the device signal shift measurements is 3.1 percent and the mean standard deviation is 3.3 milliseconds.

A fingerprint of the physical and mechanical properties of the asphalt binder can be supplied to the job-site, in terms of the device response at a range of temperatures and frequencies established at the supplier location. This fingerprint can then be compared to a fingerprint of the asphalt binder at the plant or the job-site in a rapid manner using the device to determine the fingerprints, e.g. at different frequencies. A direct comparison of in field mechanical properties of the asphalt binder with commonly tested dynamic mechanical properties of asphalt binders, e.g., shear modulus and phase angle can be performed. The device can be driven at 120 Vₚ₋ₚ AC voltage at all temperatures. The DC excitation provided in the strain gage can be as high as 15 VDC. This device produces bending strains in the order of 0.0125 percent at the center of the device when tested in air, e.g., about the maximum strain encountered. The strain levels in the asphalt binder are lower than or equal to this value, depending on the temperature of testing. The asphalt binder is expected to be within the linear viscoelastic range at these strain levels when tested with the device. The testing in air can follow the testing in asphalt, or can take place simultaneously. Strain gage data from the device can be collected after the first 10 cycles of loading the gage to avoid overheating of the piezoelectric materials due to the applied DC bridge excitation.

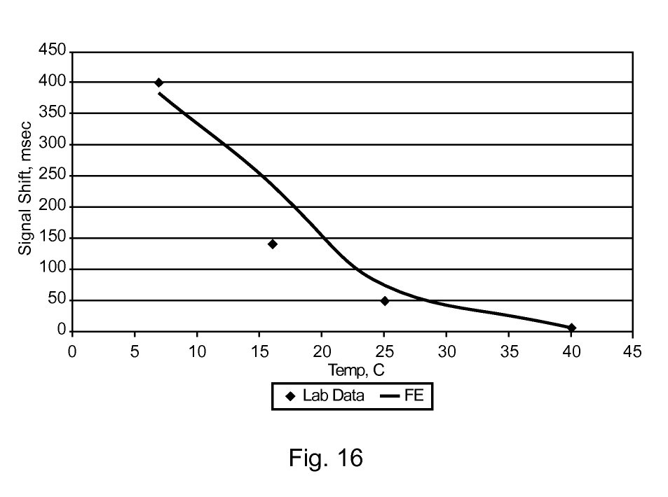

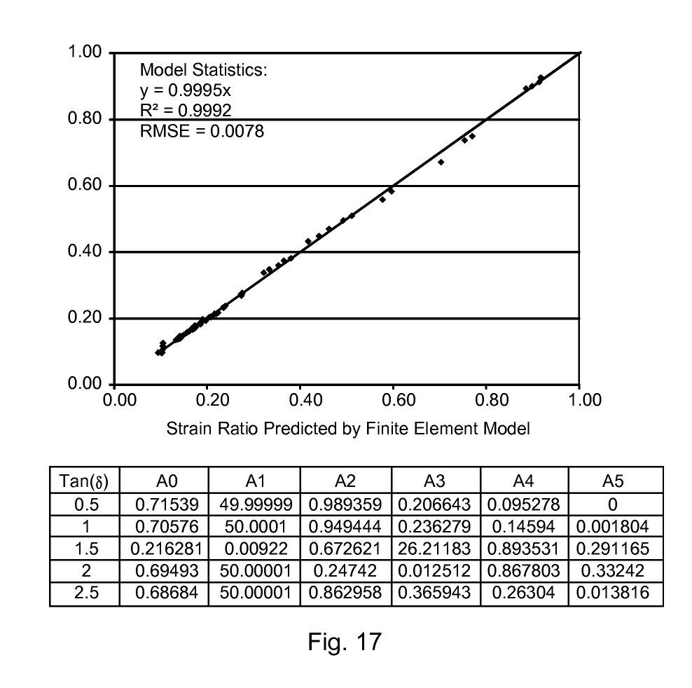

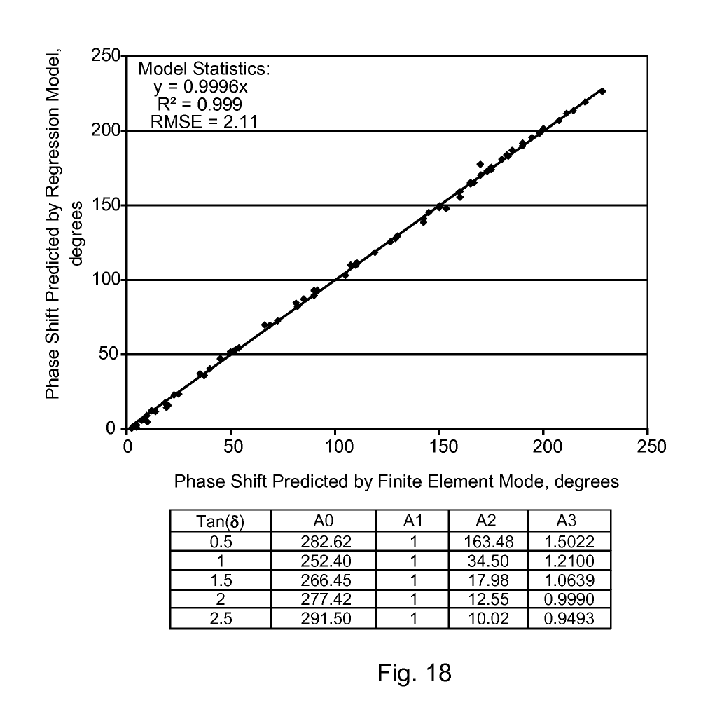

An exemplary finite element model simulates the behavior of the device in air and when embedded in asphalt. The purpose is to provide a theoretical basis for the gage's behavior. The=output of interest from each finite element analysis is the maximum bending strain at the center of the device and the associated signal shift. FE analyses can be conducted with the gage operating freely in air as well as in various asphalt binders whose properties were known (estimated from the DSR). The maximum gage bending strain outputs in asphalt can be divided by the calculated maximum gage strain output in air to obtain a strain ratio. The strain ratio from each finite element run is then compared with those determined experimentally at the same temperature and under the same loading conditions for each of the asphalt binders. The comparisons between the finite element and laboratory strain ratios for AC-10 PAV binders is shown in FIG. 15. FIG. 15 is an exemplary graph showing a correlation of a device measured strain ratio with the strain ratio estimated by a finite element model mimicking the device operation. Corresponding comparison plots for the same asphalt binders for phase shift is presented in FIG. 16. FIG. 16 is an exemplary graph showing a correlation of a device measured phase shift with the phase shift estimated by a finite element model mimicking the device operation. Using this validated FE model as a basis, several FE runs can be performed of the device operating at a frequency of 1.59 Hz in asphalt binders over a range of assumed asphalt non-dimensional stiffness M′ and δ. From each run the strain ratio and phase shift can be determined as explained in this paragraph. This database of solutions forms a basis for relating the laboratory measured strain ratio and phase shift from the device to asphalt binder G* and δ. FIG. 17 is a chart of an exemplary comparison of strain ratios from the FE model runs and the regression equations that fit the data from the finite element model runs and FIG. 18 is a chart of an exemplary comparison of phase shifts from the finite element model runs and the regression equations that fit the data from the finite element model runs. The strain ratio term can be chosen as one of the dependent variables because strains are computed in the FE-based approach used. Strain Ratio (ε_{ac}/εₐᵢᵣ)=A0*e(−A1*M′)+A2*e(−A3*M′)+A4*e(−A5*M′) presents an analytical expression relating the strain ratio and M′ for each tan(δ) value shown in FIG. 17. The goodness-of-fit statistics and plot for this expression are presented in FIG. 17. Similarly,

$$Phase Shift ( {θ}_{ac} - {θ}_{air} ) = \frac{A 0}{A 1 + A 2 * {M}^{{′ }^{A 3}}} \qquad ( 5 )$$

presents the expression relating phase shift and M′ for each tan(δ) value shown in FIG. 17. The corresponding goodness-of-fit statistics and plot for this expression are presented in FIG. 18.

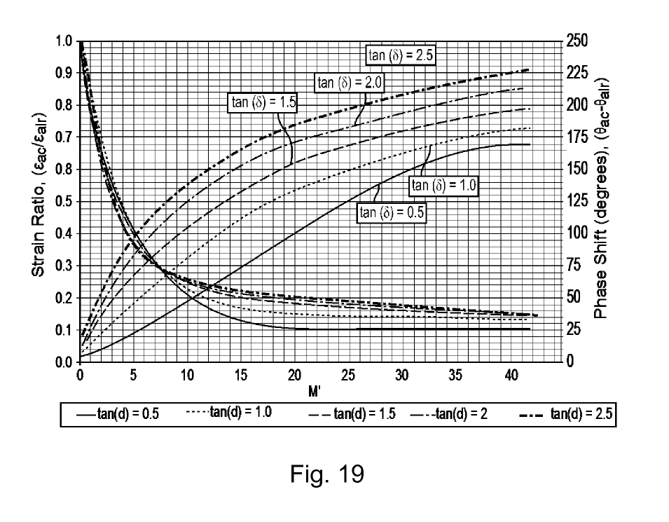

FIG. 19 is a chart of an exemplary nomograph for one of the devices showing the results of data reduction to convert, e.g., reduce, measured strain ratios and phase shifts into complex shear modulus (G*) and phase angle (δ). The nomograph is used to describe a data reduction scheme to obtain the continuum's parameters for the device. The strain at the center of the device is obtained for a given voltage and frequency of testing in air and in the continuum—εₐ and ε_{c}, respectively. The phase difference between the sinusoidal voltage input and the corresponding strain output is obtained in air as well as in the continuum—θₐ and θ_{c}. The shift angle for the medium is then determined as θ_{M}=θ_{c}−θₐ. For the given device, disk rigidity parameter D is established. Equation

$$\vert \frac{{M}_{c}}{{M}_{o}} \vert = \vert \frac{{ɛ}_{c}}{{ɛ}_{a}} \vert \qquad ( 6 )$$

can be used to determine the modified moment ratio |M_{c}/M₀|. The nomograph can be entered with the moment ratio and the shift angle used to iteratively determine the values of \hat{M}′ and tan(δ). Iteration can be performed until the differences between the respective parameters obtained from two successive steps fall within acceptable limits. Using \hat{M}′ and tan(δ) obtained,

\hat{M}′=Ê′a³/D (7)

and tan ∂=Ê″/Ê′ are used to determine E′ and E″, respectively.

The values E* and G* from

$${E}^{*} = \sqrt{{E}^{′2} + {E}^{″2}} and {G}^{*} = \frac{{E}^{*}}{2 ( 1 + {v}^{*} )} , \qquad ( 8 )$$

are determined respectively. The data reduction can be automated, e.g., using a computer program executed by a processor. In one example, DUONOMO and DUO_CALC computer programs developed by Grosz in 1995 as part of the Office of Naval Research effort to adapt the DART for marine sediment testing can be used to expedite the data reduction process. The DUONOMO program numerically constructs the nomograph for any given disk geometry and the DUO_CALC program uses this to compute \hat{M}′ and tan(δ) iteratively. The inputs to the DUO_CALC program are the measured strains and shift angles with respect to the drive signal, in air and in asphalt. These DUO_NOMO and DUO_CALC programs are available on request and can be adapted for construction of the nomographs for each device. Other programs can be developed and used.

Results of the FE-based parametric analysis of the A-Device #1C gage establish a framework to reduce the device gage responses to estimate the asphalt binder properties of interest to this research. Measured strain ratio and phase shift data from the A-Device #1C gage can be used in conjunction with FIG. 15 and the following equations:

$$\begin{matrix}Strain ratio ( {ɛ}_{ac} / {ɛ}_{air} ) = A {0}^{*} e ( - A {1}^{*} {M}^{′} ) + A {2}^{*} e ( - A {3}^{*} {M}^{′} ) + A {4}^{*} e ( - A {5}^{*} {M}^{′} ) & ( 9 ) \\ Phase Shift ( {θ}_{ac} - {θ}_{air} ) = \frac{A 0}{A 1 + A 2 * {{M}^{′}}^{A 3}} & ( 10 )\end{matrix}$$

to estimate G* and δ using the following sequence of steps: 1) enter the nomograph in FIG. 15 with known values of strain ratio and phase shift; 2) estimate an initial M′ for an assumed value of tan(δ), e.g., 1.0, based on the Strain Ration equation; 3) for the M′ estimated from step 2 and the known phase shift, estimate a new tan(δ) (this may require interpolation between the various M′ versus phase shift curves (Phase Shift equation) to estimate the tan(δ) value); 4) with the new tan(δ) value from step 3 and the known strain ratio, enter the nomograph in FIG. 15 to develop an revised estimate of M′ (as noted above); 5) iterate steps 2 through 4 as many times as needed to obtain successive estimates of M′ and tan(δ) that are within preset tolerance limits, e.g., 2 percent; and 6) estimate G′, G″ and G* using:

$$\begin{matrix}{\hat{M}}^{′} = {\hat{E}}^{′} {a}^{3} / D & ( 11 ) \\ {\hat{M}}^{″} = {\hat{E}}^{″} {a}^{3} / D & ( 12 ) \\ tan \partial = {\hat{E}}^{″} / {\hat{E}}^{′} & ( 13 ) \\ {E}^{*} = \sqrt{{E}^{{′}^{2}} + {E}^{{″}^{2}}} & ( 14 ) \\ {G}^{*} = \frac{{E}^{*}}{2 ( 1 + {v}^{*} )} . & ( 15 )\end{matrix}$$

Using the scheme described, the data reduction approach was validated for different combinations of asphalt binder G* and δ in the following table:

| | | DSR G*, | DSR δ, |
|---|---|---|---|
| | | psi (GPa) | Degrees |
| Binder Type | Test Temperature | at 1.59 Hz | at 1.59 Hz |
| AC-10 | 16° C. (61° F.) | 411 (2.8) | 61 |
| AC-10 | 25° C. (77° F.) | 66 (0.5) | 78 |
| AC-40 | 25° C. (77° F.) | 149 (1.0) | 67 |
| AC-20 PAV | 34° C. (93° F.) | 248 (1.7) | 45 |

The G* and δ shown in the table are estimated using a research grade DSR at a test frequency of 1.59 Hz.

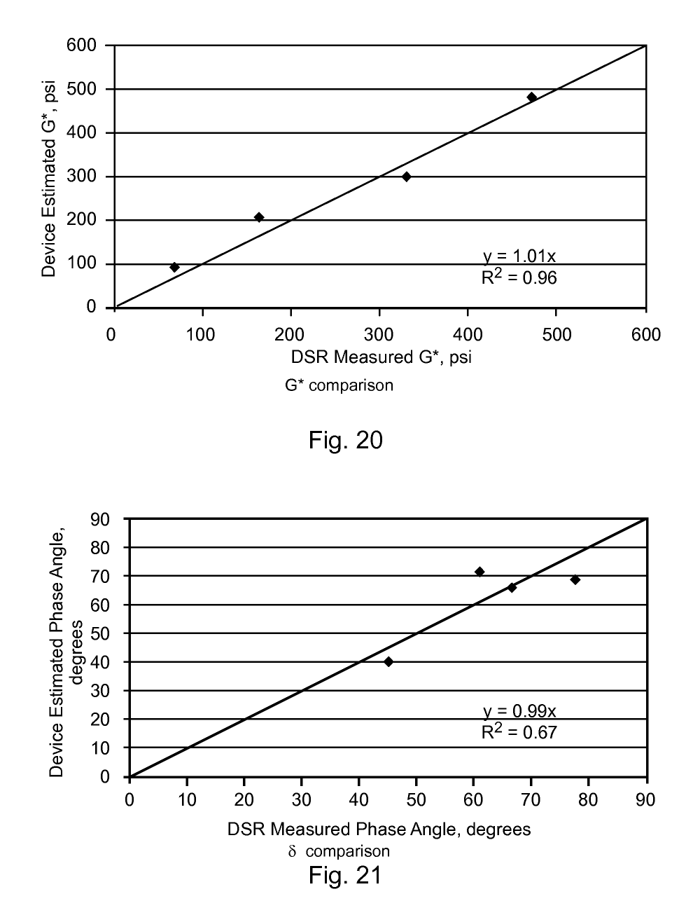

FIG. 20 presents a comparison of the DSR measured G* and the corresponding DART estimated values for the various asphalt binders evaluated. The device testing is performed using the ADART#1C gage at the same testing frequency as the DSR, i.e., 1.59 Hz. FIG. 21 presents a similar comparison for δ. Based on the data and statistics presented in the figures, an unbiased and excellent agreement can be observed between the DSR and DART G* estimates. An unbiased and fair agreement can also be observed between the DSR and DART estimates of δ—a parameter which is typically has a higher measurement error than the G*. The validation exercise can therefore establish the ability of the device and the FE-based data reduction scheme to estimate asphalt binder properties that compare well with DSR-benchmark data.

The validation exercise provides a basis for developing the framework to reduce the system, method and device gage raw outputs to determine asphalt properties of interest. Overall, both the raw gage responses—strain output and phase shift—as well as processed outputs—G* and δ—are sensitive to the properties of the surrounding medium and can be used to rapidly fingerprint asphalts in production to identify quality issues. FIG. 20 presents an example of the output from the systems, methods and devices that show sensitivity to the various grades of asphalt material. Note that the AC-10 was the softest asphalt tested as was in an unaged condition. The AC-20 (PAV) was the stiffest asphalt tested and was PAV or long-term aged. The slopes of the curves vary by binder grade and aging condition demonstrating the ability of the DART to uniquely fingerprint different binders. Further, Pen 40/50 is essentially the same binder as the AC-10 but graded using the penetration grading system as opposed to the viscosity grading system. Note that the Pen 40/50 and AC-10 fingerprints therefore line up almost on top of each other confirming that the system, method and device outputs are repeatable and the fingerprinting is unique to the binder characteristics. FIG. 21 presents the correlation between the DART estimated G* and δ parameters derived using the raw gage outputs and the data reduction framework and the DSR estimated G* and d for various asphalt binders under the same testing conditions, e.g., temperature of asphalt and frequency of DART gage excitation. FIG. 21 demonstrates the effectiveness of the systems, methods and devices to produce parameters of interest to the AASHTO M320 specification.

Given that both the raw and reduced device outputs are sensitive to the type of asphalt medium and are consistent and repeatable, the systems, methods and devices can be used in the field to fingerprint asphalt for quality assurance purposes. The device is suited for testing asphalt binders which may be produced by a supplier through the blending of different binders or through blending modifiers into a virgin binder. The blended material can be accepted as produced by the supplier in accordance with the AASHTO M320. The approved binder can be fingerprinted in the laboratory by testing it with the device system to obtain either its strain and phase shift response signature or G*-δ signature at various temperatures and frequencies as desired within the operating constraints of the device. These fingerprints can be stored and subsequently used to compare similar data obtained from device testing conducted in the field on samples taken along its journey from the supplier to the job-site. On-site blending is done often e.g., for the production of certain types of modified binders. Since the testing is low-cost and nondestructive, it can be performed on a continual basis. At the plant, where the polymer or warm-mix additive is blended in line, samples can be taken at specified intervals and tested with the device once temperature stability is obtained. Differences in the device fingerprint indicate the degree of blending compliance by comparing them with the values obtained from the approved laboratory blend. Control charts can be established to monitor the quality of the asphalt and take corrective action after additional verification is done, e.g., using the AASHTO M320 specification equipment.

The device can be used to determine the fingerprint of materials in addition to asphalt binders, e.g., a polymer, and compare the fingerprint against fingerprints for certified materials to determine a quality of the tested material. Additionally or alternatively, the fingerprint can be used to test an authenticity of the tested material against a known fingerprint of the material as determined at the supplier stage. The device can be actuated at different frequencies when submerged in the material, and an output of the strain gage of the device is processed to determine the fingerprint for the material at those frequencies. By testing the material over different frequencies, the fingerprint can be matched with fingerprints of previously tested materials, to determine a consistency of the material against specifications.

The systems, methods, devices, and logic described above may be implemented in many different ways in many different combinations of hardware, software or both hardware and software. For example, all or parts of the system may include circuitry in a controller, a microprocessor, or an application specified integrated circuit (ASIC), or may be implemented with discrete logic or components, or a combination of other types of analog or digital circuitry, combined on a single integrated circuit or distributed among multiple integrated circuits. All or part of the logic described above may be implemented as instructions for execution by a processor, controller, or other processing device and may be stored in a tangible or non-transitory machine-readable or computer-readable medium such as flash memory, random access memory (RAM) or read only memory (ROM), erasable programmable read only memory (EPROM) or other machine-readable medium such as a compact disc read only memory (CDROM), or magnetic or optical disk. Thus, a product, such as a computer program product, may include a storage medium and computer readable instructions stored on the medium, which when executed in an endpoint, computer system, or other device, cause the device to perform operations according to any of the description above.

The processing capability of the system may be distributed among multiple system components, such as among multiple processors and memories, optionally including multiple distributed processing systems. Parameters, databases, and other data structures may be separately stored and managed, may be incorporated into a single memory or database, may be logically and physically organized in many different ways, and may implemented in many ways, including data structures such as linked lists, hash tables, or implicit storage mechanisms. Programs may be parts (e.g., subroutines) of a single program, separate programs, distributed across several memories and processors, or implemented in many different ways, such as in a library, such as a shared library (e.g., a dynamic link library (DLL)). The DLL, for example, may store code that performs any of the system processing described above.

Many modifications and other embodiments set forth herein will come to mind to one skilled in the art having the benefit of the teachings presented in the foregoing descriptions and the associated drawings. Although specified terms are employed herein, they are used in a generic and descriptive sense only and not for purposes of limitation.

1. A system, comprising:
a voltage supply configured to supply a drive voltage;
a flexible device connected with the voltage supply, the flexible device configured to be embedded into a material;
a strain gage connected with the flexible device, the strain gage configured to measure a flexing of the flexible device; and
a processor connected with the strain gage, the processor configured to determine a measured strain and a phase shift between the drive voltage and the strain when voltage is supplied to the flexible device to produce a fingerprint of the material.

2. The system of claim 1, wherein the processor is further configured to convert the measured strain and phase shift into a dynamic sheer (G*) and phase angle (δ) fingerprint of the material.

3. The system of claim 1, wherein the processor is configured to compare the determined strain and phase shift to a known strain and phase shift of the material.

4. The system of claim 1, wherein the processor is configured to determine the strain and the phase shift over different frequencies of the drive voltage.

5. The system of claim 1, wherein the processor is configured to determine the strain and the phase shift over different temperatures of the material.

6. The system of claim 1, wherein the material comprises an asphalt binder.

7. The system of claim 1, wherein the flexible device is embedded into the material at a job site.

8. A method, comprising:
determining a strain of a device embedded in a material;
determining a phase shift response for the device embedded in the material; and
fingerprinting the material based on the determined strain and phase shift.

9. The method of claim 8, further comprising converting the measured strain and phase shift into a dynamic shear modulus (G*) and phase angle (δ) fingerprint of the material.

10. The method of claim 8, wherein the strain and the phase shift are determined for different frequencies.

11. The method of claim 8, wherein the strain and the phase shift are determined for different temperatures.

12. The method of claim 8, wherein the material comprises an asphalt binder.

13. The method of claim 8, further comprising comparing the fingerprint of the material against known fingerprints for the material.

14. The method of claim 8, further comprising determining a quality of the material based on the fingerprinting.

15. The method of claim 8, wherein the device is embedded at a job site.

16. A device, comprising:
a first piezoelectric layer;
a shim connected with the first piezoelectric layer;
a second piezoelectric layer connected with the shim; and
a strain gage connected with the second piezoelectric layer, the strain gage configured to measure a strain and phase shift of a material.

17. The device of claim 16, further comprising a processor, the processor configured to convert the measured strain and phase shift into a dynamic sheer (G*) and phase angle (δ) fingerprint of the material.

18. The device of claim 16, wherein the first and second piezoelectric layers comprise a PZT and the shim comprises a stainless steel.

19. The device of claim 16, wherein the strain gage outputs a measured strain when a drive voltage is supplied to the second piezoelectric layer.

20. The device of claim 19, wherein one of the first piezoelectric layer and the second piezoelectric expands and another of the first piezoelectric layer and the second piezoelectric contracts when the drive voltage is supplied.
